(19) United States
(12) Patent Application Publication
(10) Pub. No.: US 2004/0092741 A1
(43) Pub. Date: May 13, 2004

(54) Substituted pyridines via boronic acid coupling

(75) Inventors: Stephane Caron, Stonington, CT (US); Jolanta Nowakowski, Old Saybrook, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/677,064

(22) Filed: Oct. 1, 2003

Related U.S. Application Data
(60) Provisional application No. 60/425,040, filed on Nov. 8, 2002.

(51) Int. Cl. C 07D 4 3/02; C 07D 4 1/02; C07D 213/72

(52) U.S. Cl. 544/360; 546/268.1; 546/194; 546/276.4; 546/304

(57) ABSTRACT

The invention relates to a convergent process for preparing a compound of the formula (V), wherein R¹ is attached at the 2 or 3 position of the benzene ring, R² is attached at the 5 or 6 position and R¹, R² and G are as defined herein, in which an aryl boronic acid is coupled with an amine protected halo-substituted-2-aminopyridine using a palladium coupling agent. Compounds of formula V are useful as nitric oxide synthase (NOS) inhibitors in a mammal.

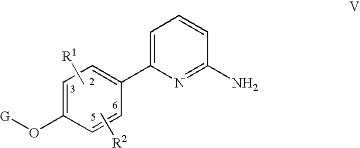

### BACKGROUND OF THE INVENTION

The present invention relates to a novel process for the preparation of 2-amino-6-(2-substituted-4-phenoxy)-substituted-pyridines. These compounds exhibit activity as nitric oxide synthase (NOS) inhibitors and are useful in the treatment and prevention of central nervous system disorders, inflammatory disorders, septic shock and other disorders.

There are three known isoforms of NOS—an inducible form (I-NOS) and two constitutive forms referred to as, respectively, neuronal NOS (N-NOS) and endothelial NOS (E-NOS). Each of these enzymes carries out the conversion of arginine to citrulline while producing a molecule of nitric oxide (NO) in response to various stimuli. It is believed that excess nitric oxide (NO) production by NOS plays a role in the pathology of a number of disorders and conditions in mammals. For example, NO produced by I-NOS is thought to play a role in diseases that involve systemic hypotension such as toxic shock and therapy with certain cytokines. It has been shown that cancer patients treated with cytokines such as interleukin 1 (IL-1), interleukin 2 (IL-2) or tumor necrosis factor (TNF) suffer cytokine-induced shock and hypotension due to NO produced from macrophages, i.e., inducible NOS (I-NOS), see Chemical & Engineering News, December 20, p. 33, (1993). I-NOS inhibitors can reverse this. It is also believed that I-NOS plays a role in the pathology of diseases of the central nervous system such as ischemia. For example, inhibition of I-NOS has been shown to ameliorate cerebral ischemic damage in rats, see Am. J. Physiol., 268, p. R286 (1995)). Suppression of adjuvant induced arthritis by selective inhibition of I-NOS is reported in Eur. J. Pharmacol., 273, p. 15-24 (1995).

NO produced by N-NOS is thought to play a role in diseases such as cerebral ischemia, pain, and opiate tolerance. For example, inhibition of N-NOS decreases infarct volume after proximal middle cerebral artery occlusion in the rat, see J. Cerebr. Blood Flow Metab., 14, p. 924-929 (1994). N-NOS inhibition has also been shown to be effective in antinociception, as evidenced by activity in the late phase of the formalin-induced hindpaw licking and acetic acid-induced abdominal constriction assays, see Br. J. Pharmacol., 110, p. 219-224 (1993). Finally, opioid withdrawal in rodents has been reported to be reduced by N-NOS inhibition, see Neuropsychopharmacol., 13, p. 269-293 (1995).

Other NOS inhibitors and their utility as pharmaceutical agents in the treatment of CNS and other disorders are referred to in U.S. Provisional Application 60/032,793, filed Dec. 6, 1996, and U.S. Provisional Application 60/014,343, filed Mar. 29, 1996.

Examples of 2-amino-6-(2-substituted-4-phenoxy)-substituted-pyridines that may be prepared in accord with the process of the present invention are disclosed in U.S. patent application Publication Ser. No. 2001/0,007,873, filed Jul. 31, 1998, and PCT International Publication Number WO 98/34919, published Aug. 13, 1998, both of which are herein incorporated by reference in their entirety. U.S. patent application Ser. No. 60/393,501, filed Jul. 3, 2002 describes novel processes for the preparation of substituted aryl boronic acids. In contrast to the processes disclosed in U.S. patent application Publication Ser. No. 2001/0,007,873, the process of the present invention is a convergent route for the preparation of 6-substituted aryl-2-aminopyridines that eliminates protection and deprotection steps, avoids intermediates that are difficult to handle and in certain embodiments enables introduction of sensitive groups such as an aminoalkyl group at an early stage, thereby eliminating additional steps for its introduction at a later stage.

### SUMMARY OF THE INVENTION

The present invention relates to a process of preparing a compound of the formula V:

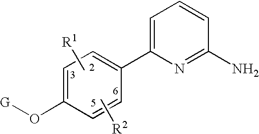

wherein in said compound of formula V the R¹ group is attached at carbon 2 or carbon 3 and the R² group is attached at carbon 5 or carbon 6 of the aryl moiety;

wherein R¹ and R² are selected, independently, from hydrogen, halo, hydroxy, (C₁-C₆)alkoxy, (C₁-C₇)alkyl, (C₂-C₆)alkenyl, and (C₂-C₁₀)alkoxyalkyl; and G is selected from hydrogen, (C₁-C₆)alkyl, (C₁-C₆)alkoxy-(C₁-C₃)alkyl, aminocarbonyl-(C₁-C₃)alkyl-, (C₁-C₃)alkylaminocarbonyl —(C₁-C₃)alkyl-, di-[(C₁-C₃)alkyl]aminocarbonyl-(C₁-C₃)alkyl-, and N(R³)(R⁴)(C₀-C₄)alkyl-, wherein R³ and R⁴ are selected, independently, from hydrogen, (C₁-C₇)alkyl, tetrahydronaphthalene and aralkyl, wherein the aryl moiety of said aralkyl is phenyl or naphthyl and the alkyl moiety is straight or branched and contains from 1 to 6 carbon atoms, and wherein said (C₁-C₇)alkyl and said tetrahydronaphthalene and the aryl moiety of said aryl may optionally be substituted with from one to three substituents, preferably from zero to two substituents, that are selected, independently, from fluoro, chloro, hydroxy, amino, (C₁-C₄)alkoxy, and (C₁-C₄)alkylamino;

or R³ and R⁴ form, together with the nitrogen to which they are attached, a piperazine, piperidine, azetidine or pyrrolidine ring or a saturated or unsaturated azabicyclic ring system containing from 6 to 14 ring members, from 1 to 3 of which are nitrogen, from zero to two of which are oxygen, and the rest of which are carbon; and wherein said piperazine, piperidine, azetidine and pyrrolidine rings and said azabicyclic ring systems may optionally be substituted with one or more substituents, preferably with from zero to two substituents, that are selected, independently, from (C₁-C₆)alkyl, amino, (C₁-C₆)alkylamino, [di-(C₁-C₆)alkyl]amino, phenyl substituted 5 to 6 membered heterocyclic rings containing from 1 to 4 ring nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from fluoro, chloro, (C₁-C₃)alkyl, (C₁-C₃)alkoxy, CF₃ and OCF₃; and wherein said piperazine, piperidine, azetidine and pyrrolidine rings and said azabicyclic ring systems may be attached to —(C₀-C₄)alkyl-O— (wherein the oxygen of said —(C₀-C₄)alkyl-O— is the oxygen atom depicted in structural formula V) at a nitrogen atom of the NR³R⁴ ring or at any other atom of such ring having an available bonding site;

or G is a group of the formula A:

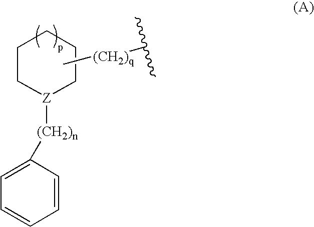

wherein Z is nitrogen or CH, n is zero or one, q is zero, one, two or three and p is zero, one or two;

and wherein the 2-amino piperidine ring depicted in structure I above may optionally be replaced with

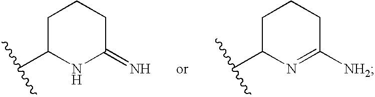

which comprises treating a compound of the formula IV:

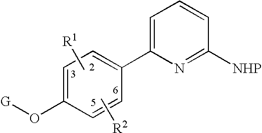

wherein R¹, R², G, R³ and R⁴ are as defined above and P is an acid removable protective group, with an acid.

In another aspect of the process of the present invention, compound IV, wherein R, R¹, R², G, R³, R⁴ and P are as defined above is prepared by treating a compound of the formula III:

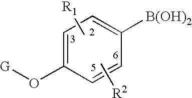

wherein R, R¹, R², G, R³ and R⁴ are as defined above, with a compound of the formula II:

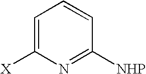

wherein P is as defined above and X is chloro, bromo or iodo in the presence of a palladium cross-coupling agent, preferably palladium acetate, more preferably tetrakis(triphenylphosphine)palladium(0).

In yet another aspect of the process of the present invention, compound II, wherein P and X are as defined above, is prepared by treating a compound of the formula I:

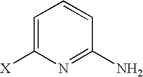

with a compound P-L, wherein P is an acid removable protective group and L is a leaving group that is replaced by the amino group of compound I, in the presence of a tertiary amine.

The term “alkyl”, as used herein, unless otherwise indicated, includes saturated monovalent hydrocarbon radicals having straight, branched or cyclic moieties or combinations thereof.

The term “one or more substituents”, as used herein, refers to a number of substituents that equals from one to the maximum number of substituents possible based on the number of available bonding sites.

The term “halo”, as used herein, unless otherwise indicated, includes chloro, fluoro, bromo and iodo.

Examples of compounds that can be prepared by the process of this invention are compounds of the formula V, and their pharmaceutically acceptable salts, wherein G is N(R³)(R⁴)(C₁-C₄)alkyl and N(R³)(R⁴) is amino, dimethylamino, methylbenzylamino, (C₁-C₄)alkylamino, di-[(C₁-C₄)alkyl]amino or one of the following groups:

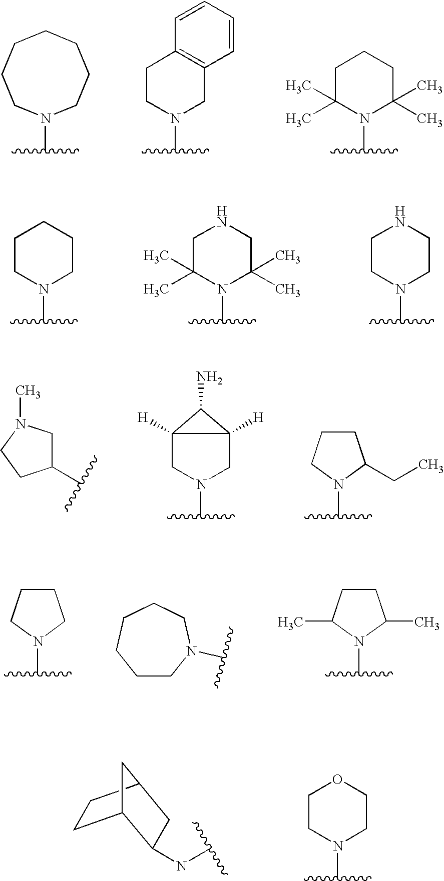

Preferred compounds of the formula V that can be prepared by the process of the instant invention include those wherein R² is hydrogen and R¹ is (C₁-C₃)alkoxy and is in the ortho position relative to the pyridine ring of formula V.

Other embodiments of this invention relate to compounds of the formula V wherein G is a group of the formula A, as defined above, wherein Z is nitrogen.

Other embodiments of this invention relate to compounds of the formula V wherein R¹ and R² are selected, independently, from (C₁-C₂)alkoxy.

Other embodiments of the invention relate to compounds of the formula V wherein G is a group of the formula A, as defined above, wherein Z is nitrogen, each of p and n is one and q is two.

Other embodiments of this invention relate to compounds of the formula V wherein the 2-aminopyridine ring depicted in formula V above, is present.

### DETAILED DESCRIPTION OF THE INVENTION

The process of the present invention and the preparation of the compounds of the present invention are illustrated in Scheme 1. The preparation of the compounds of formulas I-V are described in the Scheme and discussion that follow, wherein, unless otherwise indicated, R, R¹, R², R³, R⁴, G, X, P and L are as defined above.

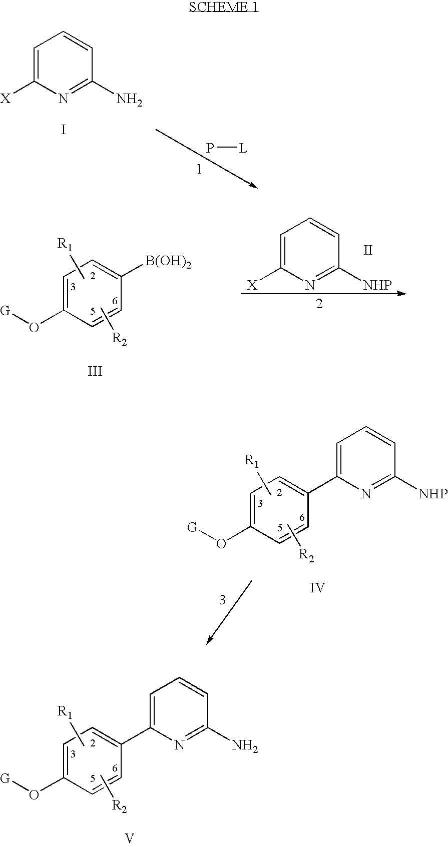

Overall the synthetic sequence of the scheme involves protection of the primary amino function of halo-substituted-2-amino-pyridine I (step 1) to form a protected 2-aminopyridine having structure II, coupling of protected 2-amino-pyridine II with boronic acid III (step 2) to form 2-amino-6-(substituted aryl)pyridine IV, treating compound IV with an acid (step 3) to form compound V wherein the protected amino group is de-protected.

In step 1 of Scheme 1 a 6-halo-2-amino pyridine, wherein halo is typically chloro, bromo or iodo, preferably bromo, is treated with P-L, a reactive amine protecting agent that can later be removed by treatment with acid, said amine protecting agent selected from C₁-C₆ aliphatic acid chloride or anhydride, arylcarboxylic acid chloride or anhydride, and other amine protecting agents known in the art, preferably (C₁-C₆)aliphatic acid chloride, most preferably acetyl chloride, in the presence of a base such as an aliphatic tertiary amine, preferably triethylamine to form amine protected pyridine II.

In step 2 of Scheme 1 phenylboronic acid derivative III is combined with amine protected pyridine II and a base such as sodium carbonate, potassium carbonate, lithium carbonate, cesium carbonate, sodium bicarbonate, potassium bicarbonate, lithium bicarbonate and cesium bicarbonate, preferably sodium carbonate and a palladium cross-coupling agent such as a palladium (C₂-C₆)carboxylate or a tetrakis(triarylphosphine)palladium(0) or mixtures thereof, preferably palladium acetate, most preferably tetrakis(tri-phenylphosphine)palladium(0) and suspended in a solvent, such as a (C₁-C₆)aliphatic alcohol, an aprotic solvent such as toluene, or water, preferably, ethanol and water. The suspension is heated at a temperature of about 25° C. to about the reflux temperature of the solvent, preferably at about reflux temperature for about 1 hour to about 12 hours, preferably about 3 hours to yield compound IV.

In step 3 of Scheme 1 protective group P is removed by treating compound IV with an acid such as hydrochloric acid or sulfuric acid, preferably HCl having an approximately 2M concentration at about 25° C. to about reflux, preferably about reflux, for about 0.5 hour to about 8 hours, preferably about 1.5 hours to yield compound V.

The present invention is illustrated by the following examples, but it is not limited to the details thereof.

### EXAMPLE 1

### N-(6-Bromo-Pyridin-2-yl)-acetamide

To a solution of 2-amino-6-bromopyridine (120.0 g, 694 mmol) in tetrahydrofuran (440 mL) was added triethylamine (137 mL, 998 mmol). The mixture was cooled in an ice bath and the temperature was maintained between 2° C. and 5° C. while acetyl chloride (122 mL, 1.72 mol) mixed with tetrahydrofuran (65 mL) was slowly added with stirring. The reaction mixture was allowed to warm to room temperature and stirring continued for 14 hours. The reaction was then quenched by addition of water (3.5 L). The precipitated product was collected by filtration and dried to afford 130.6 g (87% yield) of N-(6-bromo-pyridin-2-yl)-acetamide as a pale yellow solid, m.p. 157.4° C.

¹H NMR (CDCl₃, 400 MHz): δ=2.20 (s, 3H), 7.21 (dd, J=7.7 Hz, J=0.8 Hz, 1H), 7.56 (t, J=8.1 Hz, 1H), 8.05 (bs, 1H, NH), 8.16 (d, J=8.1, 1H).

### EXAMPLE 2

### N-{6-[4-(2-Dimethylamino-ethoxy)-5-ethyl-2-methoxy-phenyl]-pyridin-2-yl}-acetamide

Under an inert atmosphere, 5-[4-(2-dimethylamino-ethoxy)-5-ethyl-2-methoxy]-phenyl-boronic acid (127.3 g, 477 mmol), sodium carbonate (166.7 g, 1.58 mmol) and tetrakis(triphenylphosphine)palladium (2.75 g, 2.38 mmol) were suspended in ethanol (1.0 L) and water (0.1 L). The orange-brown suspension was then heated at reflux for 18 hours. The mixture was then cooled to 33° C., quenched with water (2.0 L) and extracted with two 0.5 L portions of ethyl acetate. The organic extracts were combined and washed with a 1 L portion and a 0.2 L portion of 1 M HCl. The aqueous acidic extracts were combined , cooled in an ice bath and the pH adjusted to 13 with 30% sodium hydroxide (154 mL) and the product was extracted with two 0.5 L portions of tert-butylmethylether. The extracts were concentrated to afford 166.1 g (88%) yield of N-{6-[4-(2-Dimethylamino-ethoxy)-5-ethyl-2-methoxy-phenyl]-pyridin-2yl}-acetamide as a greenish solid, m.p. 114.5° C.

¹H NMR (CDCl₃, 400 MHz): δ=1.13 (t, J=7.6 Hz, 3H), 2.02 (bs, 3H), 2.39 (s, 6H), 2.8 (q, J=7.6 Hz, 2H), 2.80 (t, J=5.9 Hz, 2H), 3.82 (s, 3H), 4.15 (t, J=5.9 Hz, 2H), 6.52 (s, 1H), 7.53 (s, 1H), 7.55 (dd, J=7.8 Hz, J=0.8 Hz, 1H), 7.69 (t, J=8.1 Hz, 1H), 8.01 (d, J=8.1 Hz, 1H), 8.69 (bs, 1H).

### Example 3

### 6-[4-(2-Dimethylamino-ethoxy)-5-ethyl-2-methoxy-phenyl]-pyridin-2-yl-amine

A solution of N-{6-[4-(2-Dimethylamino-ethoxy)-5-ethyl-2-methoxy-phenyl]-pyridin-2-yl}-acetamide (0.77 g, 2.15 mmol) in 2M HCl (15 mL) was heated at reflux for 1.5 hour. The resultant dark green solution was cooled to room temperature and extracted with tert-butylmethylether (400 mL). The aqueous layer was separated, the pH adjusted to 13-14 with 30% sodium hydroxide (4 mL) and extracted with tert-butylmethylether (50 mL). About 10 mL of tert-butylmethylether was evaporated; methylcyclohexane was added and the resultant mixture was refrigerated at about −5° C. The crystalline product that formed was collected by filtration, washed with 2 mL methylcyclohexane and dried to afford 0.6 g (88% yield) of 6-[4-(2-Dimethylamino-ethoxy)-5-ethyl-2-methoxy-phenyl]-pyridin-2-yl-amine as an off white solid, m.p. 109.4° C.

¹H NMR (CDCl₃, 400 MHz): δ=1.19 (t, J=7.6 Hz, 3H), 2.38 (s, 6H), 2.62 (q, J=7.6 Hz, 2H), 2.79 (t, J=5.9 Hz, 2H), 3.82 (s, 3H), 4.13 (t, J=5.9 Hz, 2H), 4.43 (bs, 2H), 6.39 (d, J=7.8 Hz, 1H), 6.51 (s, 1H), 7.15 (d, J=7.6 Hz, 1H), 7.44 (t, J=7.8 Hz, 1H), 7.54 (s, 1H).

1. A process of preparing a compound of the formula V having the structure: 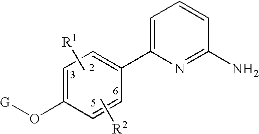
wherein in said compound of formula V the R¹ substituent is attached at carbon 2 or 3 and the R² substituent is attached at carbon 5 or 6 of the aryl moiety;
wherein R¹ and R² are selected, independently, from hydrogen, halo, hydroxy, (C₁-C₆)alkoxy, (C₁-C₇)alkyl, (C₂-C₆)alkenyl, and (C₂-C₁₀)alkoxyalkyl; and G is selected from hydrogen, (C₁-C₆)alkyl, (C₁-C₆)alkoxy-(C₁-C₃)alkyl, aminocarbonyl-(C₁-C₃)alkyl-, (C₁-C₃)alkylaminocarbonyl-(C₁-C₃)alkyl-, di-[(C₁-C₃)alkyl]aminocarbonyl-(C₁-C₃)alkyl-, and N(R³)(R⁴)(C₁-C₄)alkyl-, wherein R³ and R⁴ are selected, independently, from hydrogen, (C₁-C₇)alkyl, tetrahydronaphthalene and aryl, wherein the aryl moiety of said aryl is phenyl or naphthyl and the alkyl moiety is straight or branched and contains from 1 to 6 carbon atoms, and wherein said (C₁-C₇)alkyl and said tetrahydronaphthalene and the aryl moiety of said aryl may optionally be substituted with from one to three substituents, preferably from zero to two substituents, that are selected, independently, from fluoro, chloro, hydroxy, (C₁-C₄)alkoxy, and (C₁-C₄)alkylamino;
or R³ and R⁴ form, together with the nitrogen to which they are attached, a piperazine, piperidine, azetidine or pyrrolidine ring or a saturated or unsaturated azabicyclic ring system containing from 6 to 14 ring members, from 1 to 3 of which are nitrogen, from zero to two of which are oxygen, and the rest of which are carbon;
and wherein said piperazine, piperidine, azetidine and pyrrolidine rings and said azabicyclic ring systems may optionally be substituted with one or more substituents, preferably with from zero to two substituents, that are selected, independently, from (C₁-C₆)alkyl, amino, (C₁-C₆)alkylamino, [di-(C₁-C₆)alkyl]amino, phenyl substituted 5 to 6 membered heterocyclic rings containing from 1 to 4 ring nitrogen atoms, benzoyl, benzoylmethyl, benzylcarbonyl, phenylaminocarbonyl, phenylethyl and phenoxycarbonyl, and wherein the phenyl moieties of any of the foregoing substituents may optionally be substituted with one or more substituents, preferably with from zero to two substituents, that are selected, independently, from halo, (C₁-C₃)alkyl, (C₁-C₃)alkoxy, nitro, amino, cyano, CF₃ and OCF₃;
and wherein said piperazine, piperidine, azetidine and pyrrolidine rings and said azabicyclic ring systems may be attached to —(C₀-C₄)alkyl-O— (wherein the oxygen of said —(C₀-C₄)alkyl-O — is the oxygen atom depicted in structural formula V) at a nitrogen atom of the NR³R⁴ ring or at any other atom of such ring having an available bonding site;
or G is a group of the formula A having the structure: 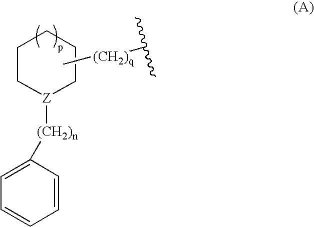
wherein Z is nitrogen or CH, n is zero or one, q is zero, one, two or three and p is zero, one or two;
and wherein the 2-amino piperidine ring depicted in structure I above may optionally be replaced with 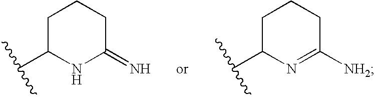
which comprises treating a compound of the formula IV having the structure: 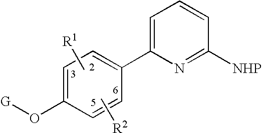
wherein R¹, R², G, R³ and R⁴ are as defined above and P is an acid removable protective group, with an acid.

2. The process of claim 1 wherein the compound of formula IV is prepared by treating a compound of the formula III having the structure: 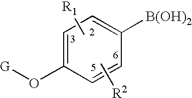
with a compound of the formula II having the structure: 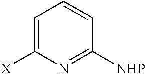
wherein X is chloro or bromo in the presence of a palladium cross-coupling agent and a base.

3. The process of claim 2 wherein the compound of formula II is prepared by treating a compound of the formula I having the structure: 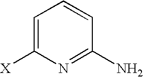
with a compound P-L, wherein P is an acid removable protective group and L is a leaving group that is replaced by the amino group of compound I, in the presence of a tertiary amine.

4. The process of claim 1 wherein the compound of formula V, wherein G is N(R³)(R⁴)(C₁-C₄)alkyl where N(R³)(R⁴) is amino, dimethylamino, methylbenzylamino, (C₁-C₄)alkylamino,

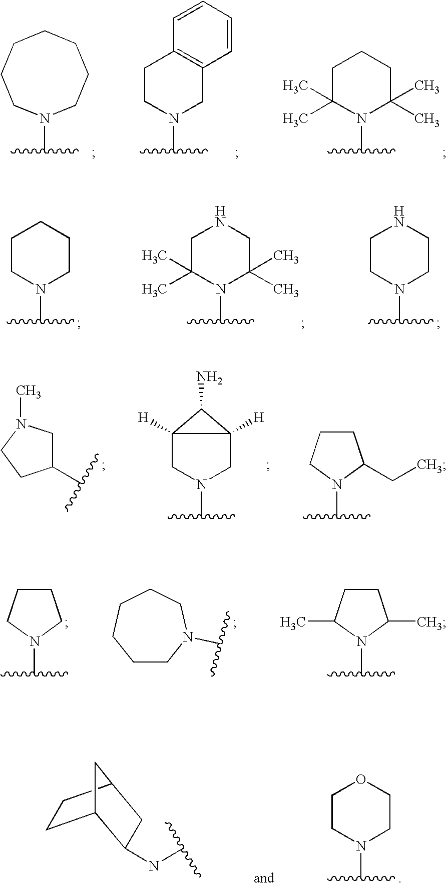

5. The process of claim 4 wherein the compound of formula IV is prepared by treating a compound of the formula III having the structure: 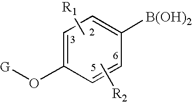
with a compound of the formula II having the structure: 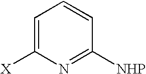
wherein X is chloro, bromo or iodo, in the presence of a palladium cross-coupling agent and a base.

6. The process of claim 4 wherein in the compound of formula V, R² is hydrogen, R¹ is (C₁-C₃)alkoxy and is in the 2 position, and in the compound of formula IV, R² is hydrogen, R¹ is (C₁-C₃)alkoxy, and R¹ is in the 2 position.

7. The process of claim 6 wherein the compound of formula IV is prepared by treating a compound of the formula III having the structure: 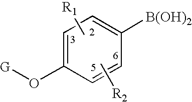
with a compound of the formula II having the structure: 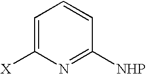
wherein X is chloro, bromo or iodo, in the presence of a palladium cross-coupling agent and a base.

8. The process of claim 2 wherein the palladium cross-coupling agent is selected from the group consisting of palladium (C₂-C₆)carboxylate, and tetrakis(triarylphosphine)palladium(0), or a mixture thereof.

9. The process of claim 5 wherein the palladium cross-coupling agent is selected from the group consisting of palladium (C₂-C₆)carboxylate, and tetrakis(triarylphosphine)palladium(0), or a mixture thereof.

10. The process of claim 7 wherein the palladium cross-coupling agent is selected from the group consisting of palladium (C₂-C₆)carboxylate and tetrakis(triarylphosphine)palladium(0), or a mixture thereof.

11. The process of claim 8 wherein the base is selected from the group consisting of sodium carbonate, potassium carbonate, lithium carbonate, cesium carbonate, sodium bicarbonate, potassium bicarbonate, lithium bicarbonate, and cesium bicarbonate, or mixtures thereof.

12. The process of claim 9 wherein the base is selected from the group consisting of sodium carbonate, potassium carbonate, lithium carbonate, cesium carbonate, sodium bicarbonate, potassium bicarbonate, lithium bicarbonate, and cesium bicarbonate, or mixtures thereof.

13. The process of claim 10 wherein the base is selected from the group consisting of sodium carbonate, potassium carbonate, lithium carbonate, cesium carbonate, sodium bicarbonate, potassium bicarbonate, lithium bicarbonate, and cesium bicarbonate, or mixtures thereof.
